(19) United States
(12) Patent Application Publication
(10) Pub. No.: US 2006/0259012 A1
(43) Pub. Date: Nov. 16, 2006

(54) PIV high pressure infusion set

(75) Inventors: Donald J. Propp, Dewitt, MI (US); Shane B. Pluta, Milford, MI (US)

Correspondence Address: Christopher J. Fildes;Fildes & Outland, P.C., Suite 2, 20916 Mack Avenue, Gross Pointe Woods, MI, 48236, US

(73) Assignee: Tri-State Hospital Supply Corporation

(21) Appl. No.: 11/128,943

(22) Filed: May 13, 2005

(51) Int. Cl. A61M 39/10; F16L 35/00; C09J 5/00

(52) U.S. Cl. 604/533; 285/332; 156/305

(57) ABSTRACT

A high-pressure tubing set includes a fitting and tubing having an end. The fitting has opposing ends and an aperture extending from one of the ends to the other end. The aperture has a tapered portion extending inwardly from one of the ends. The tubing end is received in the tapered portion and is chemically bonded thereto on an inner surface of the tapered portion, and defines a bonded portion, at a distance spaced from an end of the fitting, the end defining a beginning of the tapered portion. Fluid pressure within the tubing urges the tubing between the beginning of the tapered portion and the bonded portion outwardly, increasing the strength of the connection between the fitting and the tubing.

### TECHNICAL FIELD

This invention relates to medical infusion sets, and more particularly to high-pressure medical tubing sets suitable for use with contrast media injection using power injectors.

### BACKGROUND OF THE INVENTION

It is known in the medical field, such as in the field of radiology, to use high-pressure withstanding tubing sets to safely power-inject contrast media into patients' vascular structure via peripheral IV (PIV) catheters and infusion sets. The contrast media allows for the illumination of arteries and veins during X-rays, CAT scans, and the like. Medical device manufacturers commonly produce these tubing sets by solvent bonding extruded tubing, such as tubing made from polyvinylchloride or other resins, to injection molded end fittings. Such tubing sets are capable of accommodating the lower end portion (i.e., 100 to 300 psi) of the total power-injection pressure range of 100 to 1,400 psi.

However, due to the pooling of solvents at the junction at which the tubing is solvent bonded to the end fittings, and the resultant chemical “attack” on the tubing wall, failure of these tubing sets, in the form of bursts, may occur at these locations under elevated pressures. In an attempt to overcome these failures, manufacturers of tubing sets have increased both the tubing wall thickness and the durometer of the tubing, making the tubing much stiffer and harder to help mitigate the chemical attack by the bonding solvent. Tubing with this stiffness, however, is usually unsuitable to be left on a patient's catheter as an ambulatory extension set after leaving the radiology/X-ray/CAT-scan department. Radiology/imaging processes therefore necessitate removing the patient's ambulatory dressing, securement, and IV extension set tubing (for low pressures of 5-100 psi), temporarily using a high-pressure set for the injection of contrast media, removing the high-pressure extension set, and installing a new low-pressure IV tubing extension set, securement devices, dressing(s), antiseptics, etc. This process results in significant costs, increased nursing time, and patient disturbance.

### SUMMARY OF THE INVENTION

The present invention provides a high-pressure tubing set that can withstand fluid pressures up to approximately 500 psi without bursting and/or leaking. The thinner-walled, softer durometer, chemically attacked portion of tubing connected to an end fitting is positioned inside of at least one circumferential reinforcement member to limit leak and burst in the area of the tubing bonded to the end fitting. This allows the tubing used to be of a softer (i.e., lower) durometer and thinner wall thickness, therefore making the high-pressure tubing set of suitable flexibility to be left on the patient as a general IV extension set before, during, and after high-pressure usages such as procedures performed in radiology units. The high-pressure tubing set of the present invention can be assembled using conventional solvent bonding technology, making the high-pressure tubing set cost-effective and easy to produce.

More particularly, a high-pressure tubing set in accordance with the present invention includes a fitting and tubing having an end. The fitting has opposing ends and an aperture extending from one of the ends to the other end. The aperture has a tapered portion extending inwardly from one of the ends. The tubing end is received in the tapered portion and is chemically bonded thereto on an inner surface of the tapered portion, defining a bonded portion, at a distance spaced from an end of the fitting, the end defining a beginning of the tapered portion. Fluid pressure within the tubing during use urges the tubing between the beginning of the tapered portion and the bonded portion outwardly, increasing the strength of the connection between the fitting and the tubing.

In a specific embodiment, the tapered portion may have a truncated conical-like shape. A portion of the aperture may be wider in diameter than an outer diameter of the tubing. The tubing may be spaced from the aperture at the beginning of the tapered portion of the aperture. The tubing end may be received within the fitting a length at least that of the tubing diameter. The fitting may be tubular-like in shape. Fluid may be communicated through the tubing and fitting at pressures up to 500 psi before the fluid may leak from the tubing set.

In an alternative embodiment, a high-pressure tubing set in accordance with the present invention includes tubing having an end and a reinforcement bushing having opposing ends and an aperture extending from one of the ends to the other end. The tubing end is received in the aperture of the bushing and chemically bonded thereto about one of the ends of the bushing inside of the bushing, and defining a bonded portion, at a distance spaced from the opposite, receiving end of the bushing. The high-pressure tubing set further includes a fitting having a receiver on one end. The receiver is sized to receive the bushing. The bushing is received in the receiver and is chemically bonded thereto. Fluid pressure within the tubing urges the tubing between the bonded portion and the receiving end outwardly, increasing the strength of the connection between the reinforcement bushing and tubing.

Optionally, the aperture of the bushing may be larger in diameter than an outer diameter of the tubing. A wall thickness of the bushing may be greater than a wall thickness of the tubing. The bushing may be of a harder durometer than the tubing. The receiver of the fitting may be a bore. The fitting may include a through hole in communication with the tubing. Fluid may be communicated through the tubing and the fitting at pressures up to 500 psi before the fluid may leak from the tubing set.

A method of assembling a high-pressure tubing set includes the steps of: providing tubing having an end and a reinforcement bushing, the bushing having opposing ends and an aperture extending from one of the ends to the other end; inserting the tubing end into the aperture of the bushing; providing a fitting having a receiver on one end, the receiver being sized to receive the bushing; dipping the end of the bushing opposite the end where the tubing was inserted into a bonding solvent; and inserting the bushing into the receiver in the fitting.

Optionally, during the dipping step, the bonding solvent may only wick partially up the portion of the tubing inserted in the bushing. Further, the method may include the step of dabbing away any excess solvent prior to inserting the bushing into the receiver.

These and other features and advantages of the invention will be more fully understood from the following detailed description of the invention taken together with the accompanying drawings.

### BRIEF DESCRIPTION OF THE DRAWINGS

In the drawings:

FIG. 1 is a perspective view of a high-pressure tubing set in accordance with the present invention;

FIG. 2 is a sectional view of the high-pressure tubing set of FIG. 1;

FIG. 3 is a partial sectional view of an alternative embodiment of a high-pressure tubing set in accordance with the present invention;

FIG. 4 is a schematic diagram illustrating attachment of tubing of the high-pressure tubing set of FIG. 3 to a bushing of the high-pressure tubing set; and

FIG. 5 is a schematic diagram illustrating attachment of the bushing/tubing combination of FIG. 4 to a fitting of the high-pressure tubing set.

### DETAILED DESCRIPTION OF THE INVENTION

Referring now to the drawings in detail, numeral 10 generally indicates a high-pressure tubing set in accordance with the present invention. The high-pressure tubing set is capable of withstanding fluid pressures of up to 500 psi for applications such as contrast media injection by power fluid movers, injectors or similar, while at the same time is of suitable flexibility to be used as a low-pressure IV extension set. In the present high-pressure tubing set, the strength of the high-pressure set is achieved by moving the generally solvent-weakened attachment point/area of tubing to a fitting completely inside of, and underneath, the shelter of the fitting itself and/or the shelter of a circumferential reinforcement member.

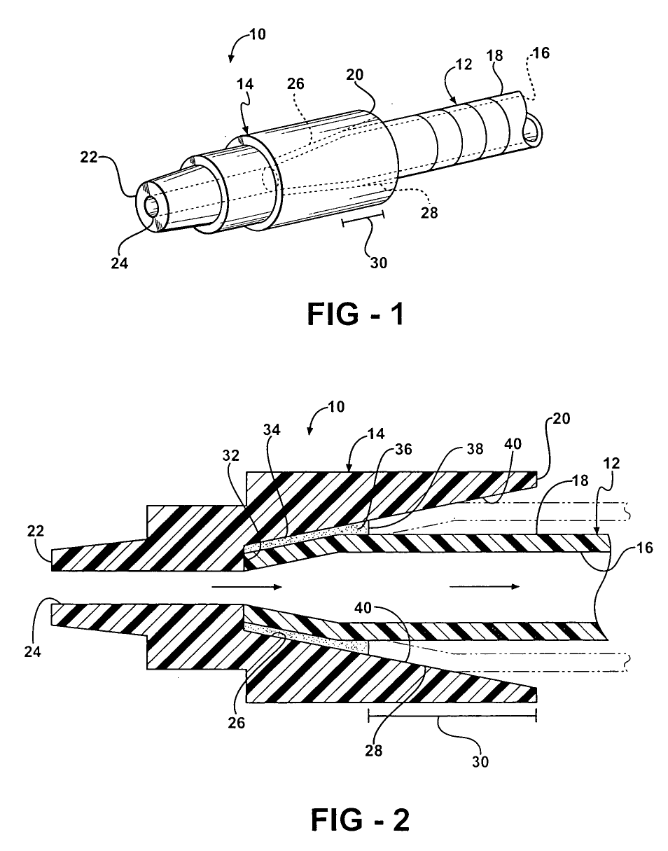

As shown in FIGS. 1 and 2, in a first embodiment of the present invention, the high-pressure tubing set 10 includes tubing 12 having an end, such as a length of medical tubing or similar, and a fitting 14. The tubing 12 has an inner wall 16 defining a tubing inner diameter and an outer wall 18 defining a tubing outer diameter. The fitting 14 has opposing ends 20, 22 and an aperture 24 extending from one of the ends 20 to the other end 22. The aperture 24 has a tapered portion 26. The tubing 12 is received in the aperture 24 and attached thereto, preferably only over some partial length of the tapered portion 26.

The tapered portion 26 of the aperture 24 may have a truncated conical-like shape. A portion 28 of the aperture 24 may be wider in diameter than the outer diameter of the tubing 12, thereby defining a gap between the tubing 12 and some portion of the tapered portion 26. The end of tubing 12 may be spaced from the aperture 24 at the end 20 of the fitting 14 where the tubing is received by the fitting. The tubing 12 may be unattached or not solvent bonded to the fitting 14 inside of the aperture 24 at a distance 30 spaced from the end 20 of the fitting. The end 20 of the fitting 14 defines a beginning of the tapered portion 26. The fitting 14 may be tubular-like in shape. The tubing 12 may be chemically bonded to the fitting 14, such as with a bonding solvent, adhesive, or similar. For example, bonding solvent or similar may be applied to the tubing 12 about an end 32 of the tubing 12 and the tubing may then be inserted into the aperture 24 of the fitting 14. The bonding solvent may primarily bond the tubing 12 to the fitting 14 at the tapered portion 26 that is smaller in diameter than the tubing. This creates a solvent witness area 34 (i.e., a bonded portion) between the tubing 12 and the tapered portion 26 of the aperture 24. Excess solvent also may pool about the area just beyond the point at which the aperture 24 becomes larger in diameter than the tubing 12, thereby creating a pooling witness area 36. The edge 38 of the pooling witness area 36 and the section of tubing 12 just beyond the edge 38 defines a weakened portion of tubing wherein the tubing wall is weakened due to chemical attack on the wall. Because this weakened portion of tubing is protected inside the aperture 24 of the fitting 14, during high-pressure usage, should the tubing 12 fail, or attempt to fail, at the weakened portion, the aperture 24 prevents leakage from the tubing set 10, as described in more detail below.

Fluid may be communicated through the tubing 12 and the fitting 14 via the aperture 24 at pressures up to 500 psi before the tubing set 10 may fail. When the tubing set 10 fails, fluid leaks from the tubing set or the tubing set may explode open causing a very loud and startling boom. During use of the tubing set 10 at pressures up to 500 psi, such as the use of the tubing set with a power fluid injector, fluid moves at high pressure (e.g., greater than 100 psi) through the aperture 24 and into the tubing 12, as shown by arrows in FIG. 2. The portion of tubing 12 inside the wide portion 28 of the aperture 24 expands such that the tubing 12 is urged outwardly and thereby pressed against the wall of the aperture 24, as shown by dashed lines in FIG. 2. This creates a self-healing/sealing area 40 between the beginning of the tapered portion 26 and the bonded portion such that should the tubing 12 leak at or about the pooling witness area 36, no fluid will leak from the tubing set 10, or at least the tubing will not “balloon” up and burst open. The sealing area 40 effectively seals the pooling witness area 36 and allows the tubing set 10 to be used at higher pressures than conventional tubing sets. This allows for the use of thinner and/or more flexible tubing walls and softer durometers, permitting continuous patient wear as described earlier.

Turning now to FIGS. 3 through 5, in an alternative embodiment of the present invention, a high-pressure tubing set 110 includes tubing 112 having an end and a reinforcement bushing 142 having opposing ends 144, 146 and an aperture 148 extending from one of the ends 144 to the other end 146. The end of the tubing 112 is received in the aperture 148 of the bushing 142 and is attached thereto some distance spaced from the end 146 of the bushing. The tubing 112 has an inner wall 116 defining a tubing inner diameter and an outer wall 118 defining a tubing outer diameter. The high-pressure tubing set 110 further includes a fitting 114 having opposing ends 120, 122 and a receiver 150 on one end 120. The receiver 150 is sized to receive the bushing 142. The bushing 142 is received in the receiver 150 and is attached thereto.

Optionally, the bushing aperture 148 may be larger in diameter than the tubing outer diameter defined by the tubing outer wall 118. A wall thickness of the bushing 142, defined by an outer wall 145 of the bushing and an inner wall 147 of the bushing, may be greater than a wall thickness of the tubing 112 defined by the tubing inner wall 116 and outer wall 118. The bushing 142 may also be of a harder durometer than the tubing 112, thereby improving the bushing's reinforcement of the tubing. The receiver 150 of the fitting 114 may be a bore. The fitting 114 may include a through hole 152 in communication with the tubing 112.

The tubing 112 may be chemically bonded to the bushing 142 and the bushing may be chemically bonded to the fitting 114, such as with a bonding solvent, adhesive, or similar as in the first embodiment. FIG. 4 illustrates a method of chemically bonding the tubing 112 to the bushing 142. First, the tubing 112 is inserted into the aperture 148 of the bushing 142 at an end 144 of the bushing until the tubing reaches the other end 146 of the bushing. Then the bushing 142 and inserted tubing 112 is dipped into a bonding solvent source (not shown) such that only an end portion 154 of the bushing is submerged in solvent 156. The end portion 154 of the bushing 142 may therefore be referred to as the dip zone. When the bushing 142 and tubing 112 are dipped in the solvent source, the solvent 156 also enters the area between the tubing 112 and the bushing aperture 148 at the dip zone 154. After, the bushing 142 and tubing 112 are removed from the solvent source, solvent 156 wicks up the area between the tubing 112 and the bushing aperture 148, creating a wick up zone 158 just beyond the dip zone 154. Adjacent the wick up zone 158, the solvent wicking ends. In this area, referred to as the self-healing/sealing zone 160, the tubing wall may be weakened due to chemical attack by the solvent 156. The portion of the tubing 112 that is bonded to the bushing 142 defines a bonded portion. Under high-pressure applications, the tubing 112 expands and presses against the bushing aperture 148 in the sealing zone 160. Should the tubing 112 fail under high pressure in this zone, the bushing 142 prevents fluid from leaking similar to the first embodiment. No solvent 156 travels beyond the sealing zone 160, therefore the area between the sealing zone 160 and the end 144 of the bushing 142 is referred to as the solvent free zone 162.

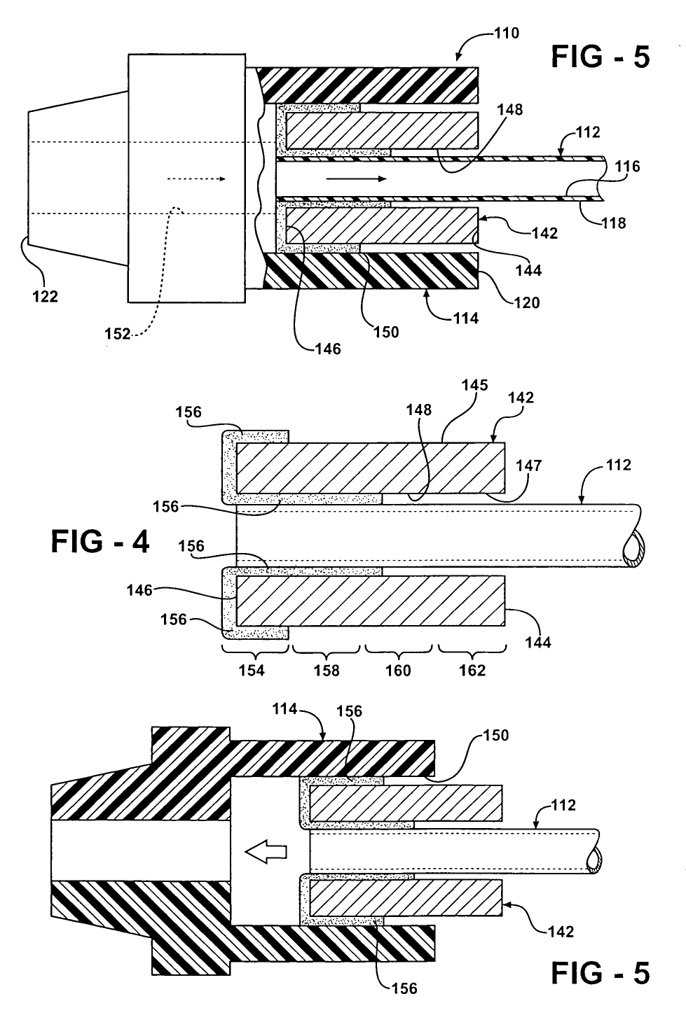

After the bushing 142 and inserted tubing 112 are removed from the bonding solvent source, any excess solvent on the exterior of the bushing may be dabbed or wiped away. Then, as shown in FIG. 5, the bushing 142/tubing 112 combination is inserted into the fitting receiver 150. The solvent 156 on the exterior of the bushing 142 bonds the bushing to the aperture 150 of the fitting 114. The fitting 114 thereby provides further reinforcement of the bushing 142 and the tubing 112.

Fluid may be communicated in either direction through the tubing 112 and the through hole 152 of the fitting 114 at pressures up to 500 psi before the fluid may leak from the tubing set 110. For reasons similar to those described in the first embodiment, the protection and reinforcement provided by the bushing 142 and the fitting receiver 150 prevent fluid leak from the tubing set 110 under high-pressure applications such as contrast media injection.

A method of assembling a high-pressure tubing set 110 first includes the step of providing tubing 112 and a reinforcement bushing 142. The bushing 142 has two opposing ends 144, 146 and an aperture 148 extending from one of the ends 144 to the other end 146, as described above. Next, the tubing 112 is inserted into an end 144 of the bushing aperture 148. Further, a fitting 114 is provided having a receiver 150 on one end 120. The receiver 150 is sized to receive the bushing 142. Then, the end 146 of the bushing 142 opposite the end 144 where the tubing 112 was inserted is dipped into a bonding solvent source. Finally, the bushing 142 is inserted into the receiver 150 in the fitting 114.

Optionally, during the dipping step, bonding solvent 156 from the bonding solvent source may only wick partially up the portion of the tubing 112 inserted in the bushing 142. Further, the method may include the step of dabbing away any excess solvent 156 prior to inserting the bushing 142 into the receiver 150.

Alternatively, a method of assembling a high-pressure tubing set 10 may first include the step of providing a length of tubing 12 and a fitting 14. As described above, the fitting 14 has two opposing ends 20, 22 and an aperture 24 extending from one of the ends 20 to the other end 22. The aperture 24 has a tapered portion 26. An end of the tubing 12 may be dipped in a bonding solvent source or similar and then may be inserted into the aperture 24. The solvent bonds the tubing 12 to the inside of the aperture 24 and the solvent stops at a boundary defined by an edge 38 of a pooling witness area 36. Therefore, an unbonded distance 30 remains between the end 20 of the fitting 14 and the edge 38 of the bonding between the tubing 12 and the aperture 24.

Although the invention has been described by reference to specific embodiments, it should be understood that numerous changes may be made within the spirit and scope of the inventive concepts described. Accordingly, it is intended that the invention not be limited to the described embodiments, but that it have the full scope defined by the language of the following claims.

What is claimed is:

1. A high-pressure tubing set comprising:
tubing having an end; and
a fitting having opposing ends and an aperture extending from one of the ends to the other end, said aperture having a tapered portion extending inwardly from one of said ends;
said tubing end being received in said tapered portion and being chemically bonded thereto on an inner surface of said tapered portion, and defining a bonded portion, at a distance spaced from an end of said fitting, said end defining a beginning of said tapered portion;
whereby fluid pressure within said tubing urges said tubing between said beginning of said tapered portion and said bonded portion outwardly, increasing the strength of the connection between said fitting and said tubing.

2. The high-pressure tubing set of claim 1, wherein said tapered portion has a truncated conical-like shape.

3. The high-pressure tubing set of claim 1, wherein a portion of said aperture is wider in diameter than an outer diameter of said tubing.

4. The high-pressure tubing set of claim 1, wherein said tubing is spaced from said aperture at the beginning of the tapered portion of said aperture.

5. The high-pressure tubing set of claim 1, wherein said fitting is tubular-like in shape.

6. The high-pressure tubing set of claim 1, wherein said tubing end is received within said fitting a length at least that of the tubing diameter.

7. The high-pressure tubing set of claim 1, wherein fluid may be communicated through said tubing and said fitting at pressures up to 500 psi before said fluid leaks from said tubing set.

8. A high-pressure tubing set comprising:
tubing having an end;
a tubular reinforcement bushing having opposing ends and an aperture extending from one of the ends to the other end;
said tubing end being received in the aperture of said bushing and chemically bonded thereto about one of the ends of said bushing inside of said bushing, and defining a bonded portion, at a distance spaced from the opposite, receiving end of said bushing; and
a fitting having a receiver on one end, said receiver being sized to receive said bushing;
said bushing being received in said receiver and being chemically bonded thereto;
whereby fluid pressure within said tubing urges said tubing between said bonded portion and said receiving end outwardly, increasing the strength of the connection between said reinforcement bushing and tubing.

9. The high-pressure tubing set of claim 8, wherein the aperture of said bushing is larger in diameter than an outer diameter of said tubing.

10. The high-pressure tubing set of claim 8, wherein a wall thickness of said bushing is greater than a wall thickness of said tubing.

11. The high-pressure tubing set of claim 8, wherein said bushing is of a harder durometer than said tubing.

12. The high-pressure tubing set of claim 8, wherein a wall thickness of said bushing is less than a wall thickness of said tubing and said bushing is of a harder durometer than said tubing.

13. The high-pressure tubing set of claim 8, wherein the receiver of said fitting is a bore.

14. The high-pressure tubing set of claim 8, wherein said fitting includes a through hole in communication with said tubing.

15. The high-pressure tubing set of claim 8, wherein fluid may be communicated through said tubing and said fitting at pressures up to 500 psi before said fluid leaks from said tubing set.

16. A method of assembling a high-pressure tubing set, said method comprising the steps of:
providing tubing having an end and a reinforcement bushing, said bushing having opposing ends and an aperture extending from one of the ends to the other end;
inserting said tubing end into the aperture of said bushing;
providing a fitting having a receiver on one end, said receiver being sized to receive said bushing;
dipping the end of said bushing opposite the end where the tubing was inserted into a bonding solvent; and
inserting said bushing into the receiver in said fitting.

17. The method of claim 16, wherein during the dipping step, the bonding solvent only wicks partially up the portion of said tubing inserted in said bushing.

18. The method of claim 16, including the step of dabbing away any excess solvent prior to inserting the bushing into the receiver.
